(19) United States
(12) Patent Application Publication
(10) Pub. No.: US 2003/0045781 A1
(43) Pub. Date: Mar. 6, 2003

(54) Device for processing signals for medical sensors

(76) Inventor: Michael N. Rosenheimer, Guenzelhofen (DE)

Correspondence Address: ST. ONGE STEWARD JOHNSTON & REENS, LLC, 986 BEDFORD STREET, STAMFORD, CT, 06905-5619, US

(21) Appl. No.: 10/214,663

(22) Filed: Aug. 8, 2002

(30) Foreign Application Priority Data
Aug. 13, 2001 (DE) 101 38 799.7

(51) Int. Cl. A61B 5/00

(52) U.S. Cl. 600/300

(57) ABSTRACT

The invention relates to a device for processing the signals of medical sensors for display or interpretation, respectively, in data monitors. The sensor signals are initially scaled or corrected, respectively, by means of an active circuit and are stored if and when necessary. The signals so corrected are then signalled to the data monitor by means of a bridge simulator or supplied to a correction circuit that corrects the signals of the sensor as such by feeding additional voltages or currents, respectively, or also by connecting further impedance elements.

This application claims priority of pending German Patent Application No. 101 38 799.7 filed on Aug. 13, 2001.

### FIELD OF THE INVENTION

The present invention relates to a device for processing signals originating from medical sensors in such a way that the latter may be displayed or analyzed, respectively, by standardized data monitors.

Such sensors are used outside the human body or are implanted in the human body, for example for pressure gauging, temperature detection or also for measuring the oxygen saturation.

Medical sensors and particularly sensors adapted for implantation in the human and also in the animal body must be completely sterilized prior to their application. This involves high demands on the sensors as such and on all parts fixedly connected to them, such as connecting cables or even connectors. Moreover, these sensors must be particularly simple to handle without faults because after implantation they are no longer accessible. Apart there from, it must be possible to handle them rapidly and properly even under a time pressure because these sensors are often used on emergency patients or at least in the course of surgical operations where limited time is available only.

The sensor signals are mostly displayed with so-called patient monitors—which will be briefly referred to as monitors. In typical cases, such a monitor comprises the appropriate means for signal processing and amplification as well as for displaying the sensor signals.

The typical structure and the co-operation of a sensor monitor system should be illustrated here in an exemplary manner with reference to a standard pressure sensor. Temperature sensors or even other sensors present, of course, an analog design.

The typical pressure sensor consists, for example, of a semiconductor sensor that varies its resistance in response to the application of pressure. In order to achieve a better de-coupling of disturbance parameters, specifically improved temperature stability, such sensors are connected in a half-bridge or full-bridge circuit (Wheatstone bridge). These bridges require a voltage supply for power feed and then furnish an output signal that is proportional to the product of the measured value and the supply voltage. Such pressure sensors can be designed in such a small size that they can be implanted into the body without any problems. For a connection to the environment, these sensors are mostly provided with a thin connecting line that leads to an electrical connector. The sensor, the connecting line and the connector must be completely sterilized prior to their implantation. Common sterilizing methods are the gas sterilization (e.g. ETO), the plasma sterilization (e.g. with hydrogen peroxide) and autoclaving (steam at a high pressure). Specifically the method mentioned last is widely common in hospitals because it ensures a high degree of sterility whilst it is simple to manage. In that method, the sensor is exposed to a high pressure and to high temperatures. This method involves high demands on the materials used.

The monitor is now connected via a connecting cable, which realizes often an adaptation to the sensor-specific connector pin configuration, with the sensor. The monitor comprises means for feeding the bridge circuits of the sensor. Such supply voltages are mostly in the range of ±5 Volt up to ±10 Volt so that the sensor may also deliver correspondingly high sensor output signals. High signal amplitudes are expedient because they improve the noise immunity. Moreover, different feed signal waveforms are known. For example, one part of the monitors supplies the sensors with a continuous D.C. voltage. This permits the simplest analysis because a continuous sensor signal is obtained. Other sensors operate on chopped supply units because, on the one hand, they reduce the power consumption of the monitor as such, which is important in operation on batteries, for example, and, on the other hand, they reduce the power consumption of the sensor so that the temperature drift of the sensor can be reduced. Moreover, monitors are known which supply the sensors with an a.c. current.

On principle, a great number of sensors may be combined with these monitors because the bridge circuits, being passive components, which are used in the sensors, are independent of the supply voltage. For an exact adaptation it is often only necessary to adapt them to the connector pin configuration and in a few cases an additional provision of resistors in the circuit is required. The most important prerequisite for compatibility is, as a matter of fact, an appropriate sensitivity. As a standard in patient monitors in medical engineering, a sensitivity of 5 μVN/mmHg has been generally accepted for pressure sensors. It is possible, for example, to employ specific resistive sensors operating on a supply voltage from roughly 7.5 to 10 Volts in order to achieve such sensitivity. These sensors are, however, comparatively expensive. When sensors of essentially lower costs are used, which can be operated only on a maximum supply voltage of roughly 1.5 Volt, a correspondingly lower sensitivity is achieved. For this reason, matching with or adaptation to the current monitors is no longer possible.

Prior to the application of the pressure sensor offset correction or offset value storage is required because the monitor needs affixed reference point. Particularly in the case of implantable sensors, this offset correction must mostly be made prior to implantation because an implanted sensor is no longer accessible. A pressure sensor that can still be balanced after implantation is described in the German utility model G 94 20 576.0. There, the sensor membrane proper is relieved from the environmental pressure via a pneumatic system for offset correction. When this pneumatic system cannot be employed for reasons of space or when another type of sensor such as a temperature sensor must be employed the offset correction operation is indispensable prior to implantation.

For offset correctional sensor is connected to the respective monitor and a zero point situation is produced on the sensor directly. Such a zero point situation is, for example, a defined temperature in the case of a temperature sensor or the environmental air pressure in the case of a pressure sensor. Then this zero point situation is measured on the monitor and the measured value is stored as offset value. Subsequently, the implantation is carried out. For a further use the sensor must be connected to that monitor exclusively that had been used for offset correction. A useful measurement is not possible with other monitors not storing the offset value information in their memories.

Another problem in the application of implantable sensors is the functional check or calibration, respectively, in the implanted condition. In this respect, a circuit published in the U.S. Pat. No. 4,760,730, for example, provides a remedy. There, a calibration unit is used, which is connected between the monitor and the sensor, for delivering a defined signal to the monitor. The sensor as such, however, is not checked. In view of this fact the efficiency of this calibration unit is extremely doubtful.

### SUMMARY OF THE INVENTION

The present invention is based on the problem of providing a device for processing the signals from medical sensors, which may be used also for the application of less expensive sensors whose sensitivity differs from the standardized sensitivity. Moreover, it is also envisaged that even during the application of these sensors, for instance after implantation or after application underneath a bandage or a plaster, various monitors may be connected, with the possibility to perform new offset correction operations with these monitors, without access to a sensor being required.

One inventive solution to this problem is defined in the independent Patent claims. Improvements of the invention are the subject matters of the dependent claims.

The inventive device comprises an active measuring circuit that is provided for scaling or correction of the sensor signals. This circuit will mostly consist of an amplifier that raises the low signal amplitudes of the sensor to higher amplitudes easier to process. Moreover, this amplifier or a following amplifier stage may be used to carry out amplitude scaling in such a way that the amplitudes are raised to a standardized value. This signal is now used to control a bridge simulator that simulates a full-bridge or a half-bridge. A connected monitor measures the values of this bridge. Hence the bridge simulator simulates the properties of a sensor whose characteristics may vary from those of the sensor type actually employed. When, for example, a less expensive sensor with a sensitivity of only 1/10 of the expensive pressure sensors is used instead of the usually employed expensive pressure sensors this may be compensated with a gain of 10 in the amplifier stage. This increased sensitivity is then signaled to the monitor by means of the bridge simulator. Hence, from the viewpoint of the monitor, a pressure sensor with the standardized sensitivity is connected.

In addition to the scaling of the sensor values it is, of course, also possible to compensate an offset, to compensate the temperature, with temperature values of a separate temperature sensor being additionally considered, for instance, or even to compensate a non-linear characteristic. Moreover, even sensors may be employed which cannot be connected as a bridge circuit, for example because they furnish a signal-dependent output voltage.

With such an inventive device it is now possible to connect different monitors even after the implantation of the sensor. A repeated offset correction operation is not required because the offset correction and optionally the scaling or a more complex correction of the measuring signal is or are carried out by means of the inventive system.

In accordance with the invention, moreover an active measuring circuit is provided for scaling or correction of the sensor signals. This circuit, however, does not control a bridge simulator, like in the previously described case, but it takes an influence on the real bridge of the sensor by connecting additional impedances or by feeding additional voltages or currents, respectively. In this way, the monitor corrects the sensor measurement by means of additional values in this embodiment.

For example, a correcting signal of a correction value generator may be coupled via a resistor to one or both outputs of the bridge circuit. Hence, an offset error can be corrected by the addition of a constant value. When a temperature-dependent voltage is added it is also possible to achieve temperature compensation. Moreover, the addition of a correcting value may also take place in the bridge supply. As the bridge output signal is proportional to the product of the measured variable and the supply voltage, the measured variable can preferably be scaled or the amplification gain can preferably be corrected via the supply voltage.

Apart there from, the impedance of individual bridge branches can be corrected for a correction of the bridge by means of controlled resistors such as FET elements. In the case of non-linear bridges, the correction may also be carried out as a function of actually measured values. The correcting values applied to this end may be optionally determined from a correction memory or by means of an appropriate correcting circuit.

Moreover, the compensation may be carried out by means of connected resistors or resistor networks, preferably by means if digital-to-analog converters.

In another expedient embodiment of the invention, the bridge simulator comprises controllable resistors. In the simplest case, these resistors include motor potentiometers, for example. Electronically controllable resistors such as field effect transistors are substantially better because they are faster and they do not require maintenance. In these elements the resistance of the drain-source channel is a function of the gate voltage. Hence, this voltage is set for control of the resistance. More complex circuits constituted by several semiconductors are equally conceivable, however, which simulate a resistance behavior by control or appropriate feedback control. A digitally controlled resistor network is employed with particular preference. Such controlled resistor networks are commercially available, for example, by the designation “electronic potentiometer”. The application of digital-to-analog converters is particularly preferred. Such converters are provided with a digitally controlled resistor network so that a bridge circuit can be simulated in a particularly simple and low-cost manner.

Another expedient embodiment of the invention operates with at least one multiplier for bridge simulation. As normal bridge circuits furnish an output signal proportional to the product of a measured variable and the bridge supply voltage this multiplication can also be simulated by a multiplier. To this end, this multiplier multiplies a value derived from the bridge supply voltages by a second value derived from the sensor signal. Analog multipliers or even digital multipliers may be used for the multiplying function, for example. An analog-to-digital converter constitutes a special case of a digital multiplier. However, in this case the wiring is slightly different from the wiring in the previously discussed case. In the previous case, the connected resistor network of the digital-to-analog converter is used exclusively. There, the supply current of the monitor flows through the resistor network. The measured value is tapped at the output of this resistor network. When the resistor network of the digital-to-analog converter is used as multiplier it is supplied indirectly by the supply voltage of the monitor. Hence, in the simplest case, a voltage may be derived, by means of a voltage divider, from the supply voltage of the monitor for the supply of the resistor network. As a matter of fact, it is also possible that the supply voltage of the monitor is detected by means of an analog-to-digital converter, multiplied in a digital multiplier, for example in a micro controller, by the measured value, and is finally output by means of a digital-to-analog converter to the monitor. In any case, the measured value is output as voltage or current value rather than in the form of impedance or an impedance ratio in this embodiment of the invention.

In another embodiment of the invention, the sensor as such is also supplied by the active circuit. Hence, an adaptation to different supply voltages of the sensors is possible. When, for example, the monitor is intended for a bridge supply voltage of 10 Volt it may destroy the sensor, which is designed for lower voltage, when the monitor is connected to the sensor directly. Therefore, a conversion or adaptation to voltage values permitting an expedient operation of the sensor is carried out in the active circuit.

Another embodiment of the invention comprises at least one memory for storing at least one offset value or for storing scaling or correcting values, respectively. Such memories may be implemented as digital memories in correspondence with prior art, for instance in the form of a non-volatile EEPROM or even as analog memories. A micro controller preferably controls the memory.

In another expedient embodiment of the invention, a non-contacting, preferably telemetric connection is provided at an optional site between the sensor as such and the bridge simulator. Such a connection may be implemented, for instance, by the transmission of the signals by means of electromagnetic waves. Here, the transmission by radio or even infrared signals is particularly expedient. Such telemetric connections had not been possible in prior art so far available when sensors were intended for use in a bridge circuit with the common monitors.

According to a further embodiment of the invention, a controller is provided to monitor the offset correction operation. This controller monitors the offset value drift of the sensor after the supply voltage has been turned on or after the sensor has been connected. The output value of the sensor will mostly approach a settled value in an asymptotic manner. The controller now monitors this approximation. To this end, for instance the variation of the sensor signal per unit of time may be analyzed. When the value drops below a threshold once or over a defined period of time one may assume, for instance, that the settled value has been reached. Only when this settled value has been reached the offset correction function is enabled. This provision prevents, on the one hand, the measurement of the offset value when after a transient period the settled value is not yet reached, or, on the other hand, a offset value measurement under non-constant environmental conditions such as the movement of a pressure sensor. After enabling the offset value measurement may be triggered via a starting signal. Such a starting signal may be given, for example, via the connector from an external contact or an external voltage source, respectively, or even from an external data stream from a signaling input or the telemetric path, respectively, i.e. by radio or infrared signals. It is equally possible to issue the starting signal also by a magnetic field sensor such as magnetic-field dependent resistors or even reed contacts by approaching or withdrawing a magnet. The starting signal may optionally also be issued by varying or alternating magnetic or electric fields detected by appropriate sensors.

In parallel with this design, optionally a time window may be defined for offset correction. For example, after the settled value has been reached one could activate the release for offset correction only for a period of 10 seconds in order to preclude erroneous offset correction that could result in cancellation of the previous offset value.

With this embodiment of the monitoring feature, the sensor signal is monitored directly. Hence, a malfunction of the sensor can be detected with a comparatively high probability. This monitoring function is preferably carried out by means of a micro controller.

In a further expedient embodiment of the invention, a controller is provided that signals a zero point value to the monitor. The demands for zero point value signaling may be issued, for example, by the previously described signaling means or even under time control within a specified interval after connection of the sensor or after start of the supply voltage, respectively. These and other controllers mentioned in this document preferably contain a micro controller.

According to another embodiment of the invention, a controller is provided that signals calibration values to the monitor. The demands for calibration value signaling may be issued, for example, by the previously described signaling means or even under time control within a specified interval after connection of the sensor or after start of the supply voltage, respectively.

In correspondence with another embodiment of the invention, a controller is provided that signals zero point values and calibration values to the monitor in alternation. The demands for signaling of these values may be issued, for example, by the previously described signaling means or even under time control within a specified interval after connection of the sensor or after start of the supply voltage, respectively. It is optionally possible as well to signal the values directly after the start of the supply voltages or after connection of the sensor, respectively, until the sensor is ready for operation and has reached the settled state after a transient period. As a result, the user or the connected monitor is informed when the sensor is actually ready for operation and measurements can be performed. This embodiment of the invention permits a functional control of the sensor function, which is substantially deeper and the more informative than this is the case in prior art, which entails decisive advantages specifically in medical engineering, particularly in emergency situations. For example, the readiness for operation is preferably signaled only after a complete functional check of the sensor by the controller. Hence the user can be certain that not only the cable or the connectors are in a proper condition but also that the sensor as such operates properly. Apart there from, the varying offset value values or calibration values may be used to set the monitor or to check the monitor functions.

In another embodiment of the invention, a controller is provided in such a form that it is designed for checking the sensor signals. For example, important operating parameters of the sensor can preferably be monitored. Examples of such parameters are the power consumption of the sensor, the plausibility check of the sensor output values or even the detection of additional parameters such as temperature measurement in the case of a pressure sensor. Moreover, the controller is so designed that it signals a fault condition. This signaling function can optionally be implemented via an additional connecting line, a telemetric path, by audible means or even by visual signals, using additional signaling means.

In another expedient embodiment of the invention, a micro controller or a microprocessor is used for control or monitoring, respectively. The respective functions for scaling or for offset correction can then be implemented in this micro controller with simple arithmetic operations. It is equally possible, in the simplest manner, to set up a correction table for correcting non-linearity of sensors, which furnishes the sensor correction values to the microcomputer. Additionally, a plurality of different sensor parameters can be stored in a memory associated with the micro controller. In such a memory, correction tables or even scaling factors of different sensor types or even the individual values of individual sensors can be stored.

### BRIEF DESCRIPTION OF THE DRAWINGS

In the following, the invention will be explained in more details with reference to the drawings wherein:

FIG. 1 is the block diagram of an inventive device;

FIG. 2 shows the block diagram of an inventive device in the case of application of a telemetric connection;

FIG. 3 is a detailed block diagram of an inventive device;

FIG. 4 illustrates time-based diagrams for a clear representation of the functions of the controller;

FIG. 5 shows an example of the implementation of a half-bridge simulator with electronically controllable resistors, and

FIG. 6 illustrates an example of the implementation of a half-bridge simulator with electronically controllable resistors.

### DETAILED DESCRIPTION OF THE DRAWINGS

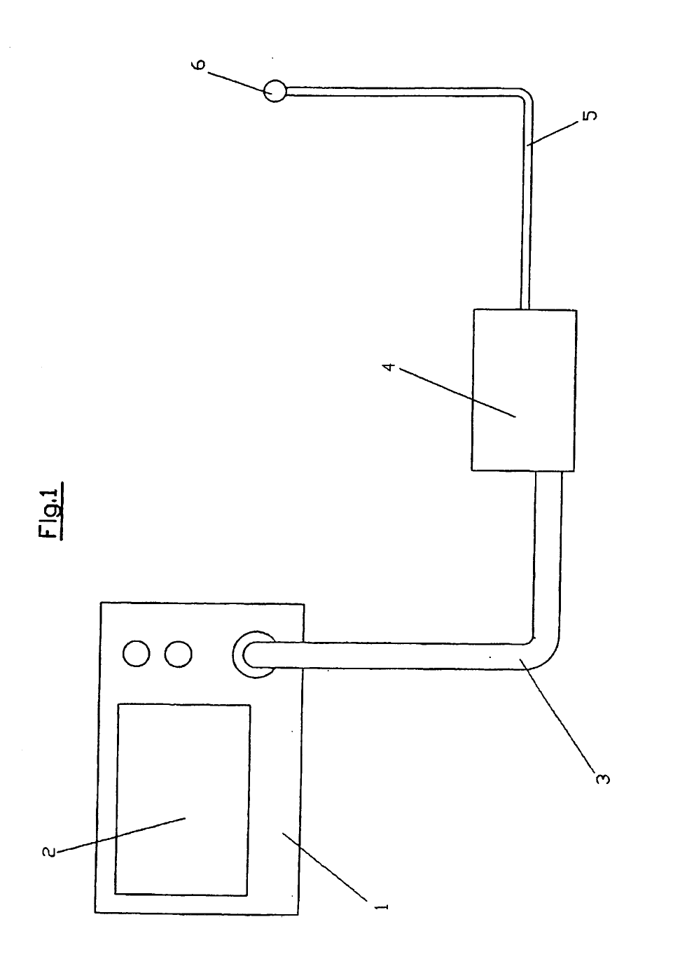

The simplified block diagram of an inventive device is illustrated in FIG. 1. A sensor (6), which may be a temperature sensor, a pressure sensor or a sensor detecting other parameters to be measured, for instance, furnishes signals by means of a sensor connecting line (5) to a device for processing signals (4). This device is mostly applied outside the body but in special designs it may also be implanted. The sensor as such may optionally also be integrated into the device for signal processing. The processed signals are then transmitted to the monitor (1) for a further analysis or for display on a display unit (2) by means of a connecting line (3) that provides for adaptation to different connector systems and is therefore also referred to as compatibility cable.

For the sake of clarity, here only a single sensor is illustrated. It is also possible, of course, to connect several sensors.

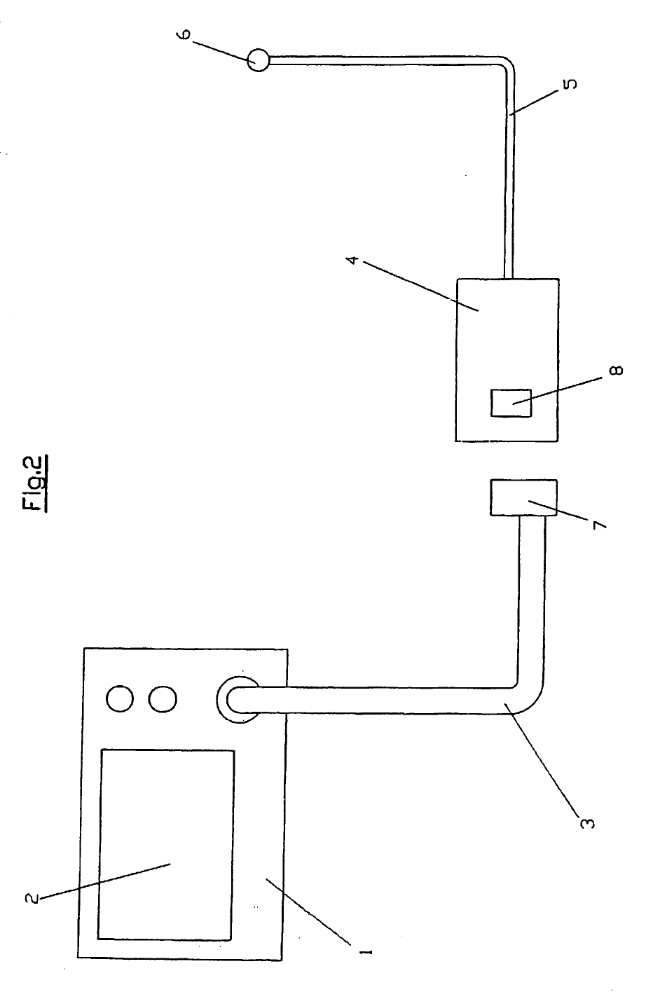

FIG. 2 shows the simplified block diagram of an inventive device for the case of application of a telemetric connection. Here, too, the signal of the sensor (6) is transmitted to a device for signal processing (4) by means of an optical sensor connecting line (5). This processing device includes a telemetry adapter (8) that is preferably designed for emission of the measured values and optionally also for receiving control commands as well as other data or for emission of calibration data and other information such as status information. This telemetry adapter (8) communicates with a telemetry-connecting unit (7) that is connected to the monitor by means of the connecting cable (3).

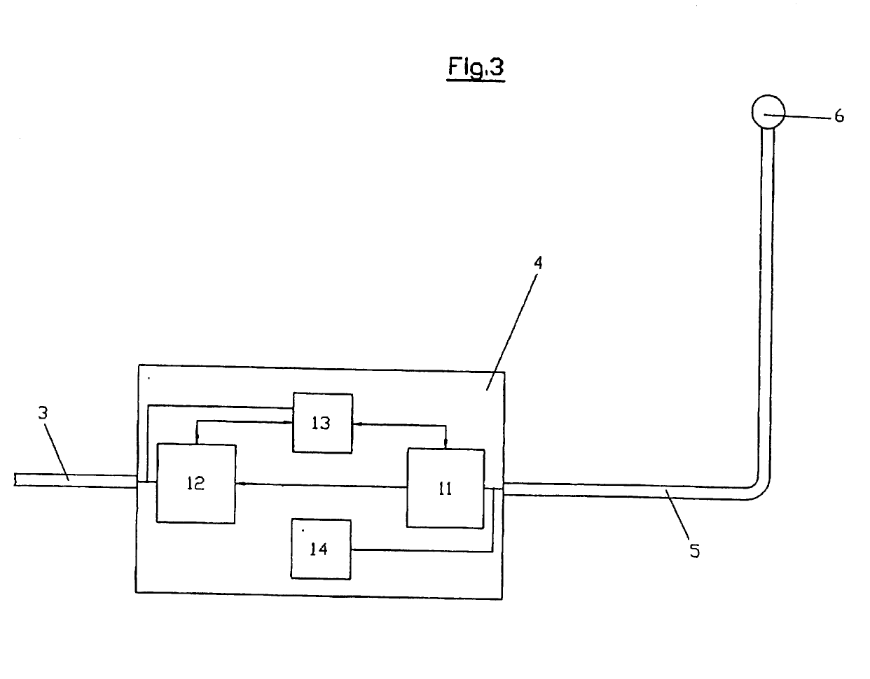

FIG. 3 shows a more detailed view of an example of the structure of an inventive device for signal processing (4). The signals of the sensor (6) are processed by means of a sensor signal processor (11) that carries out the amplification or correction of the sensor signal. The sensor signal so amplified or corrected, respectively, is passed on to a bridge simulator (12). The latter simulates to a monitor, which is connected via the connecting line (3), the behavior of a bridge circuit reflecting the corrected values of the sensor. An optional controller (13) takes optionally an influence on the sensor signal processor (11), the bridge simulator (13), while it is optionally connected to the monitor, preferably for signaling or even only for feeding. Moreover, an optional feeder means (14) is provided as feeder for the sensor (6).

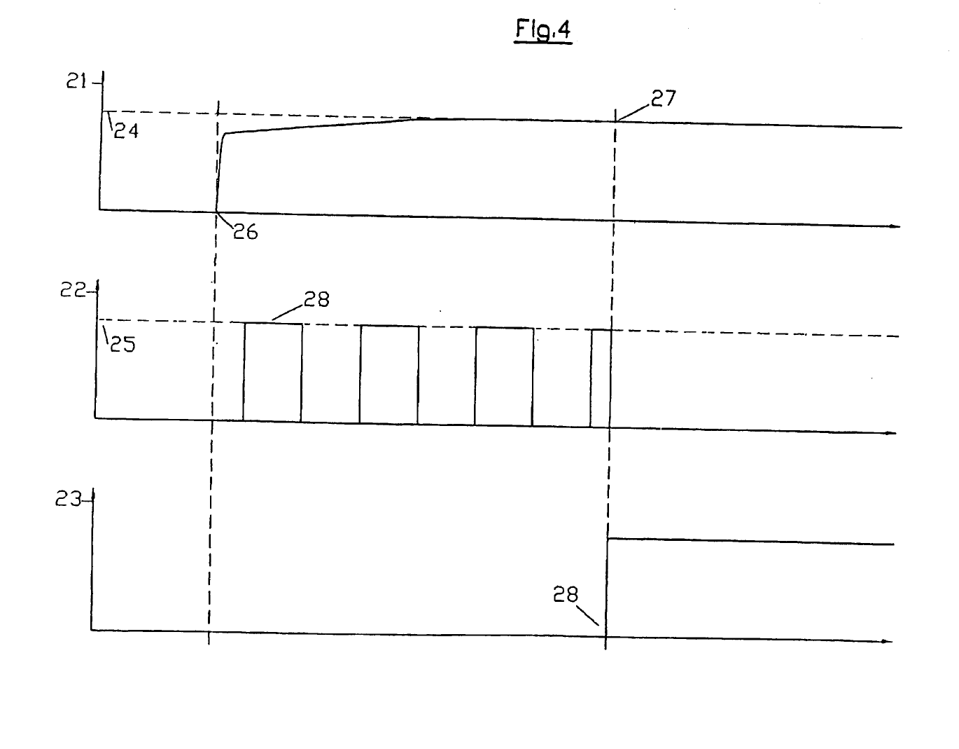

FIG. 4 shows three exemplary time-based diagrams to illustrate the functions of the controller. The horizontal axis is the time axis in all three diagrams. The vertical axis (21) of the top diagram indicates the magnitude of the sensor signal. The vertical axis (22) in the middle diagram indicates the magnitude of the signal transmitted via the bridge simulator to the monitor. Finally, the vertical axis (23) of the bottom diagram illustrates a digital signal enabling the offset correction function.

In the deactivated state, all the signals are preferably in an idle condition, for example at a zero current value. When now the inventive device is connected or started, respectively, by the point of time (26) the sensor signal first rises rapidly and approaches a settled value (24) in an asymptotic manner. The approximation within a predetermined limit to this value takes place by the point of time (27). The fact that this value is reached is detected, for instance, by an interpretation of the pitch of the graph and by detection of the situation that the pitch drops below a minimum value. In the interval between these two points of time, the inventive device issues optionally alternating signals to the monitor. These signals alternate between a low state, which signals a zero value, and the issuance of a calibration value (28) with a predetermined amplitude (25). The issuance of these values ends preferably simultaneously with the point at which the threshold level of the sensor signal is reached. With this provision, this condition is signaled to the monitor or the user, respectively. Simultaneously with the point at which this threshold is reached, an enable signal is issued for offset correction. From that point of time onwards, an offset correction cycle can hence be performed.

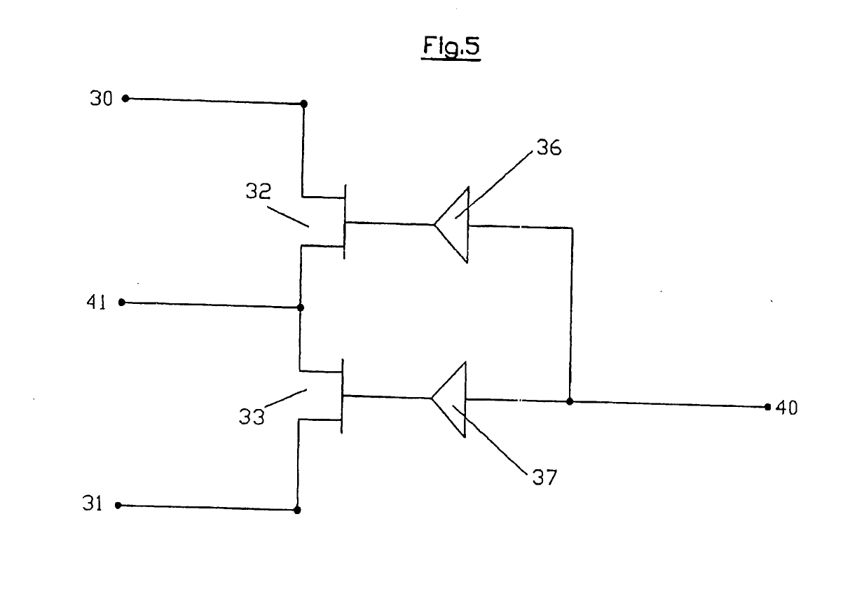

FIG. 5 illustrates an example of the simulation of a half-bridge circuit by means of electronically controllable resistors in the form of field effect transistors. The monitor ensures the feeding of the bridge simulator by means of the terminals (30, 31). Different impedance elements in the bridge branches are simulated by field effect transistors (32, 33). These field effect transistors are controlled by means of the controller circuits (36, 37). The set value is determined via a common terminal (40). The output signal issued to the monitor is output via the terminal (41).

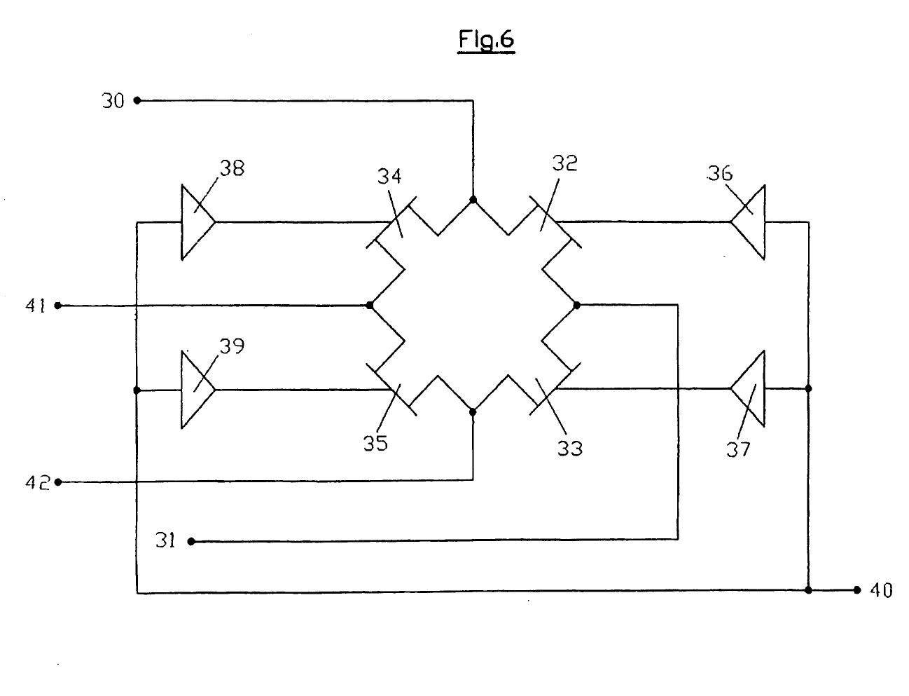

FIG. 6 illustrates an example of the simulation of a full-bridge circuit by means of electronically controllable resistors in the form of field effect transistors. Here, the same reference numerals as in FIG. 5 are used. Here merely four field effect transistors (32, 33, 34, 35) are provided which are controlled by the corresponding controller circuits (36, 37, 38, 39). Moreover, two output signals are issued to the monitor via the terminals (41, 42).

1. A device for processing signals of medical sensors (6) for display or analysis in a data monitor (1) that is designed for the analysis of signals from full-bridge or half-bridge circuits, comprising:
a measuring circuit (11) for the detection, for scaling or correction of the sensor signals, and
a bridge simulating circuit (12) comprising electrically controllable resistors,
said resistors being connected to said data monitor to simulate either a scaled or corrected bridge circuit or both for the data monitor.

2. A device for processing signals of a medical sensor (6) for display or analysis, respectively, in a data monitor (1) that is designed for the analysis of signals from full-bridge or half-bridge circuits, comprising:
said medical sensor having a bridge-circuit directly connected to a data monitor, and
a memory unit for storing at least one of an offset value, a scaling value, and a correction value, and
electrically controlled resistors which are connected in parallel with said medical sensor which is controlled by said memory unit to simulate either a scaled or corrected bridge circuit or both for the data monitor.

3. The device according to claim 1 or 2, wherein said electrically controllable resistors comprise at least one of a FET, a controlled resistor network, and a digital-to-analog converter.

4. The device according to claim 1 or 2, wherein an analog multiplier is provided for bridge simulation.

5. The device according to claim 1, wherein the electrical power for the medical sensor is supplied by said measuring circuit.

6. The device according to claim 2, wherein the electrical power for the medical sensor is supplied by said memory circuit.

7. The device according to claim 1 or 2, wherein a non-contacting telemetric connection is provided between said sensor and said device for processing signals.

8. The device according to claim 1 or 2, wherein a controller is provided for monitoring the offset correction process, said controller enabling the offset correction function within a predetermined time window only after a settled condition of the sensor signal has been reached.

9. The device according to claim 1 or 2, wherein an external offset value and configuration memory is provided which is adapted to be connected by telemetry or by wire.

10. The device according to claim 1 or 2, wherein a controller is provided for signalling the offset value to said monitor, with the offset value being signalled in response to control signals or within a predetermined time interval after the sensor has been connected to the data monitor or after electrical power has been applied.

11. The device according to claim 1 or 2, wherein a controller is provided for signalling calibration values to said monitor, the calibration values being signalled in response to control signals or within a predetermined time interval after the sensor has been connected to the data monitor or after electrical power has been applied.

12. The device according to claim 1 or 2, wherein a controller is provided for alternating signalling zero point values and calibration values, to said monitor, said values being signalled in response to control signals or within a predetermined time interval, respectively, after the sensor has been connected to the data monitor or after electrical power has been applied or until the sensor is ready for operation or after a settled condition of the sensor signal has been reached.

13. The device according to claim 1 or 2, wherein a controller is provided for monitoring the sensor function, by monitoring important operating parameters or output values of the sensor and signalling a malfunction of the sensor.

14. The device according to claim 1 or 2, wherein a micro controller or a microprocessor is used for controlling or monitoring the sensor functions.
